(19) United States
(12) Patent Application Publication
(10) Pub. No.: US 2026/0130852 A1
(43) Pub. Date: May 14, 2026

(54) BIPHASIC CHRONO-ADAPTIVE NUTRACEUTICAL SYSTEM FOR ENHANCED CIRCADIAN BIOAVAILABILITY

(71) Applicant: AETHERION GLOBAL LLC, LEWES, DE (US)

(72) Inventor: Ramon E. Gutierrez-Sandoval, Concepcion (CL)

(73) Assignee: AETHERION GLOBAL LLC, LEWES, DE (US)

(21) Appl. No.: 19/211,207

(22) Filed: May 18, 2025

Related U.S. Application Data
(60) Provisional application No. 63/720,739, filed on Nov. 14, 2024.

(51) Int. Cl. A61K 9/107; A61K 9/1271; A61K 9/51; A61K 31/05; A61K 31/12; A61K 31/122; A61K 31/197; A61K 31/685; A61K 31/7034; A61K 36/02

(52) U.S. Cl. CPC A61K 9/1075; A61K 9/1271; A61K 9/5161; A61K 31/05; A61K 31/12; A61K 31/122; A61K 31/197; A61K 31/685; A61K 31/7034; A61K 36/02

(57) ABSTRACT

A biphasic nutraceutical system designed to deliver GRAS-certified bioactive compound families across two circadian-aligned phases: a daytime formulation supporting metabolic activation and cognitive readiness, and a nighttime formulation facilitating nutritional regeneration and immune balance. Active compounds are encapsulated respectively within phospholipid-stabilized micellar vesicles for sublingual delivery and pH-sensitive polysaccharide hydrogel matrices for delayed absorption. This dual-phase structure enables non-invasive, phase-specific release and enhances nutrient bioavailability without invoking pharmacological mechanisms. The system integrates principles of nutritional chronobiology and adaptive timing to synchronize absorption with physiological receptivity windows. Exclusively composed of GRAS-aligned functional ingredients, it supports general biological function under non-clinical conditions, remaining fully within the regulatory scope of dietary supplements under 21 CFR § 170.30.

### CROSS-REFERENCE TO RELATED APPLICATIONS

This application is a utility patent application claiming priority to U.S. Provisional Patent Application Ser. No. 63/720,739 filed Nov. 14, 2024, which is incorporated by reference herein in its entirety.

### BACKGROUND OF INVENTION

### 1) Field of Invention

The present invention pertains to the field of biotechnology and nutraceutical delivery systems. More specifically, it concerns a dual-phase formulation designed to align bioactive compound administration with circadian physiological rhythms, enabling structured support for metabolic, immunological, and regenerative functions without invoking therapeutic mechanisms.

### 2) Description of Related Art

Comparative Patent Landscape and Claim Differentiation—PLPC-NX introduces a dual-phase nutraceutical platform designed to synchronize nutrient absorption with circadian metabolic windows. This delivery strategy is distinct from prior formulations that lack timing-specific bioavailability alignment or structurally defined phase division. The system integrates a biphasic release mechanism with differentiated encapsulation strategies phospholipid micelles for daytime uptake and polysaccharide nanoparticles for sustained evening release. These features are not disclosed in any single prior reference, and their combination reflects a non-obvious integration of known components.

The table below highlights key differentiators versus representative technologies cited in the prior art:

TABLE
Comparative Patent Landscape and Structural Differentiation

| | Bio- | | Distinctive |
|---|---|---|---|
| Patent/ | availability | Limitation in | Element of |
| Technology | Strategy | Prior Art | PLPC-NX |
| US20180071272A1 | Lipid-based | No synchroniza- | Phase-specific |
| (Nanoemulsion | nano- | tion with | timing guided |
| System) | emulsifica- | circadian markers | by chrono- |
| | tion | | alignment |
| US20190284567A1 | Polymeric | Not adapted to | Structured |
| (Hydrogel Delivery) | sustained | AM/PM partition | biphasic release |
| | release | or biomarker | tuned to |
| | | input | biological cycles |
| U.S. Pat. No. | Broad- | No structural | Functional phase |
| 8,017,147B2 | spectrum | division by time | separation with |
| (Antioxidant Blend) | ingredient | or delivery phase | bioactive |
| | mix | | mapping |

These comparisons demonstrate that while prior systems individually disclose encapsulation, known ingredients, or daily supplementation strategies, no reference teaches their integrated use in a two-phase, circadian-aligned delivery architecture with timing-controlled modulation of nutrient release. As such, PLPC-NX presents a claimable distinction based on structural configuration, temporal logic, and adaptive delivery-all absent in the cited art

### Experimental Validation of Chrono-Adaptive Delivery Efficiency

To substantiate the system's structural performance, PLPC-NX has undergone a series of non-clinical validation protocols including in vitro, ex vivo, and computational modeling of nutrient delivery behavior.
Caco-2 enterocyte assays demonstrated a 3.8-fold increase in transcellular uptake (p<0.01), confirming improved bioactive assimilation through phospholipid micellar transport.
LC-MS/MS enzymatic degradation profiling indicated a 42% reduction in compound breakdown, supporting greater integrity of bioactives during digestive transit and hepatic interface exposure (p<0.05).
Dynamic simulation of gastrointestinal release confirmed a biphasic absorption profile aligned with metabolic windows, with nutrient availability peaking at 7±1 hours (day phase) and 10±2 hours (night phase). These simulations model nutrient interaction timing within digestive and intercellular environments without invoking systemic distribution assumptions.
   All experimental methods remain within non-therapeutic boundaries and are designed to evaluate delivery efficiency, nutrient retention, and absorption timing consistent with dietary supplement frameworks. No pharmacokinetic endpoints were assessed or claimed.

General Technical Description and Structural Integration and Circadian Synchronization Summary—The present invention relates to the field of nutraceutical biotechnology and introduces PLPC-NX (Cellular Optimization Bioactive Phospholipoproteomic Complex), a biphasic nutritional support system designed to align nutrient delivery with circadian metabolic cycles. The system is engineered to provide functional modulation of physiological processes through structured composition and non-therapeutic delivery mechanisms.

PLPC-NX is composed exclusively of GRAS-classified bioactive compounds, including phospholipids, adaptogens, antioxidants, and regulatory peptides. These are partitioned into two complementary formulations: a morning-phase composition intended to support metabolic activation, cellular energy generation, and immune preparedness; and an evening-phase composition supporting regenerative processes, immune stabilization, and oxidative recovery. Both phases are encapsulated using phase-specific carriers-micellar phospholipid vesicles for daytime delivery and biodegradable polysaccharide nanoparticles for nighttime release.

The composition is defined not by therapeutic endpoints but by the structural alignment of compound families with physiological timing. Bioavailability is enhanced through microfluidization and vesicle stabilization techniques, supporting transcellular absorption and resistance to enzymatic degradation. Observed retention time and absorption profiles confirm functional synchronization with biological receptivity windows, without invoking pharmacological effects.

Nutritional Delivery Method—Circadian-Aligned Biphasic Administration Another aspect of the invention is directed to a method for delivering bioactive compounds via a circadian-aligned biphasic nutraceutical system. The method includes:
providing a first composition formulated with antioxidant adaptogens and phospholipid-associated nutritional cofactors, encapsulated within micellar lipid transport vesicles to support enhanced absorption;
administering said first composition in the early diurnal phase, within approximately 2 hours of waking;
providing a second composition comprising regenerative-phase bioactives embedded within pH-sensitive polysaccharide nanoparticles;
administering said second composition in the pre-nocturnal phase, aligned with typical circadian transition toward rest-phase metabolism;
modulating the timing and proportion of both compositions through a non-therapeutic chrono-metabolic synchronization algorithm designed to align nutritional release profiles with general physiological patterns.
   Administration is configured via time-release oral capsules or gastro-resistant formats, including enteric-coated layers with differential disintegration. The synchronization algorithm may reference user-specific indicators such as heart rate variability or salivary cortisol levels, used in wellness monitoring frameworks. pH-responsive matrices are configured to disintegrate under intestinal pH conditions below 5.5. For users with elevated physiological demands, the system may be adapted to support recovery-related nutritional requirements, without implying therapeutic or pharmacological effects.
   Biphasic Nutraceutical System with Circadian-Modulated Sublingual and Hydrogel Delivery

One embodiment of the present invention relates to a biphasic nutraceutical system designed for circadian-aligned support of immune, metabolic, and regenerative functions. The formulation comprises two structurally distinct phases, each encapsulated in a specialized delivery matrix optimized for specific circadian windows.

### First-Phase Composition—Sublingual Liposomal Format

The first-phase composition includes phosphatidylcholine-class lipids, stilbenoid-class polyphenols, adaptogenic phenolic glycosides, immunomodulatory polysaccharide fractions, and mitochondrial coenzyme quinones. It is formulated as a phospholipid-stabilized liposomal emulsion for sublingual administration in liquid or sachet format. This non-invasive route enables rapid mucosal diffusion, bypassing gastric degradation and hepatic first-pass metabolism, and improving systemic nutrient availability without invoking pharmacological mechanisms.

### Second-Phase Composition—Hydrogel-Based Delayed Release

The second-phase composition includes phosphatidylserine-class lipids, carotenoid-class antioxidants, non-proteinogenic amino acid analogs, polyphenolic diketones, long-chain omega fatty acids, and peptide-based adaptogens. It is embedded in a pH-sensitive polysaccharide-polymer hydrogel matrix configured for buccal or oral absorption aligned with nocturnal metabolic cycles. Disintegration occurs at pH<5.8, with optional co-lyophilization using tocopherol-class antioxidant stabilizers.

### Regulatory Framework and GRAS Compliance

Both compositions are formulated exclusively with GRAS-recognized functional compound families and exclude any synthetic pharmacological agents. The system is structurally optimized for phase-specific, non-invasive nutritional delivery synchronized with circadian physiological cycles, and is compliant with the regulatory scopes of:
U.S. GRAS (21 CFR § 170.30),
EU Novel Food Regulation (2015/2283),
Canada's NHP classification.

Personalized Adaptability and Modular Implementation—Formulations may be adaptively configured across user populations based on demographic or physiological parameters such as age, inflammatory status, and stress biomarkers. Claim protection encompasses sublingual emulsions, hydrogel matrices, and bifurcated unit-dose packaging, as well as embedded biometric modulation via CMSA.

Structural Integrity and Claim Scope Summary—The invention is structurally defined by:
A bifurcated circadian architecture (morning/evening),
Class-specific bioactive encapsulation,
Format-aligned release logic (sublingual vs delayed),
Full modularity and personalization capacity, All within the scope of non-pharmaceutical dietary supplement classification.

Structural Configuration and Micellar Stability—The first-phase micelles, formed by high-pressure homogenization, exhibit particle sizes between 80-150 nm and zeta potential between −18 and −22 mV. These characteristics support colloidal stability and enhanced transcellular permeability. The hydrogel matrix maintains release consistency and stability during buccal or oral deployment.

Integrated System Integrity and Non-Combinability—The PLPC-NX system is a structurally interdependent delivery platform. Its dual-phase design exhibits emergent nutrient retention and biomarker alignment that are not replicable through mechanical combination of conventional encapsulation technologies, time-based dosing, or isolated functional ingredients.

Functional Interlock Between Phases—The timing, release kinetics, and physiological integration of each phase depend on activation of the counterpart. Independent administration or substitution disrupts chrono-aligned efficacy and nullifies systemic performance. This interlock is governed by the embedded CMSA logic and cannot be achieved through prior art assemblies.

The internal Chrono-Metabolic Synchronization Algorithm (CMSA) functions as a non-optional gating system, regulating nutrient release according to biometric indicators such as HRV variability, salivary cortisol patterns, and IL-6 fluctuations. No conventional delivery platform-including AM/PM systems, sustained-release capsules, or single-phase nanoemulsions-incorporates a comparable adaptive control logic.

Empirical data confirm a non-linear enhancement in nutrient retention and circadian alignment not anticipated by conventional pharmacokinetic projection. The dual-phase encapsulation and CMSA coordination generate an emergent delivery behavior that invalidates functional equivalence by substitution or mechanical replication.

Accordingly, substitution of any core element—including encapsulation topology, timing logic, or biometric-gated modulation—with generic mono-phase or sustained-release mechanisms results in systemic desynchronization and loss of intended functionality. This delivery architecture cannot be reconstructed from known technologies without inventive step, and thus qualifies as structurally and operationally non-obvious under 35 U.S.C. § 103.

Synergistic Interaction and Non-Reconstructability The combined performance of the PLPC-NX system results from emergent, non-linear interactions among encapsulation architecture, circadian-phase timing, and biometric-modulated release logic. Attempts to replicate this behavior by combining individual components from prior art-such as encapsulation formats, active ingredients, or AM/PM dosing strategies-fail to produce the observed synchronization or biomarker-aligned outcomes.

These properties arise not from ingredient selection alone, but from their co-encapsulation topology, structural interlock, and algorithmically governed timing-none of which are taught or suggested in any cited reference, whether individually or in combination.

For detailed mapping of claims to figures, descriptive anchors, and technical differentiators, see Annex A

### BRIEF DESCRIPTION OF THE DRAWINGS

FIG. 1 is a high-level conceptual diagram of the biphasic circadian-aligned nutraceutical framework, showing the general relationship between the daytime and nighttime phases, along with their respective physiological targets.

FIG. 2 illustrates a human-centered schematic view of circadian modulation, indicating nutrient delivery pathways that activate energy metabolism, cognitive function, immune priming, cellular regeneration, and detoxification during the appropriate biological windows.

FIG. 3 presents a sinusoidal chart representing baseline circadian rhythms, overlaid with the synchronized nutrient release curves of the biphasic delivery system at two peaks: morning and evening.

FIG. 4 depicts the general flow of nanoencapsulated bioactives from ingestion to cellular uptake, highlighting intestinal passage, transcellular lipid transport, and intracellular integration.

FIG. 5 provides a labeled cross-section of a phospholipoproteomic nanoparticle, showing its bilayer phospholipid membrane, stabilization shell, encapsulated payload, and structural scale at nanometer resolution.

FIG. 6 is a comparative absorption efficiency bar graph among four systems: standard capsules, hydrogel-based systems, liposomal carriers, and PLPC-NX, indicating relative uptake percentages and enzymatic resistance.

FIG. 7 illustrates the core components of the PLPC-NX platform, divided into: (1) Bioactive pool, (2) Dual-phase delivery vehicle, and (3) Circadian-tuned modulation logic, with directional flows of nutrient processing.

FIG. 8 is a schematic representation of the Chrono-Metabolic Synchronization Algorithm (CMSA), illustrating a non-therapeutic modulation framework that includes input variables reflecting general circadian indicators (e.g., rest-activity cycles, autonomic variability), an internal phase-alignment engine, and an output logic module that governs the timing and amplitude of nutraceutical compound availability in alignment with predefined chrono-nutritional parameters.

FIG. 9 shows a standardized administration sequence over 24 hours, combining tablet or liquid formats, with overlay curves of cortisol and melatonin dynamics to guide intake timing.

FIG. 10 is a schematic overview of the biphasic circadian-aligned nutraceutical system, illustrating the dual-phase framework wherein daytime components support energy metabolism, cognitive function, and immune activation, and nighttime components support cellular regeneration, detoxification, and neuroprotection.

FIG. 11 is a circular systems diagram showing the chrono-adaptive biphasic delivery mechanism, incorporating dual-layer nanoparticle structures, synchronized metabolic rhythms, and phase-specific modulation of immune and metabolic responses.

FIG. 12 is a cross-sectional structural depiction of a phospholipoproteomic nanoparticle, illustrating its bilayer lipid membrane, nanoencapsulated core, and inner payload compartment, with an overall diameter of approximately 200 nanometers.

FIG. 13 is a release profile graph showing the temporal kinetics of biphasic compound release over a 24-hour period, demonstrating two peak bioavailability phases corresponding to circadian metabolic receptivity windows.

FIG. 14 is a flowchart diagram of the Chrono-Metabolic Synchronization Algorithm (CMSA), outlining the input biomarker signals, internal adaptive modulation logic, and the output regulation of nutrient release timing and amplitude.

FIG. 15 is a schematic of the circadian dosing protocol, showing administration times for morning and evening phases, superimposed with sinusoidal curves representing endogenous hormonal rhythms.

FIG. 16 is a block diagram of the modular components comprising the PLPC-NX system, including the nutritional agent (102), the biphasic delivery module (104), and the chrono-adaptive control module (106).

FIG. 17 is a comparative absorption graph displaying the difference in systemic bioavailability between the biphasic PLPC-NX system and a conventional liposomal formulation, measured over a 24-hour interval.

FIG. 18 is a cellular interaction diagram depicting the non-pharmacodynamic intracellular action of biphasic compounds, including nanoparticle docking, membrane integration, and downstream nutritional support within the target cell.

FIG. 19 shows a rule-based schematic 995 of the Chrono-Metabolic Synchronization Algorithm (CMSA), incorporating general physiological inputs (e.g., HRV, cortisol:melatonin ratio) and executing a logic sequence to determine the optimized timing of nutraceutical compound release without invoking pharmacodynamic or diagnostic effects. The schematic illustrated a non-therapeutic logic structure for time-aligned compound release based on general circadian indicators.

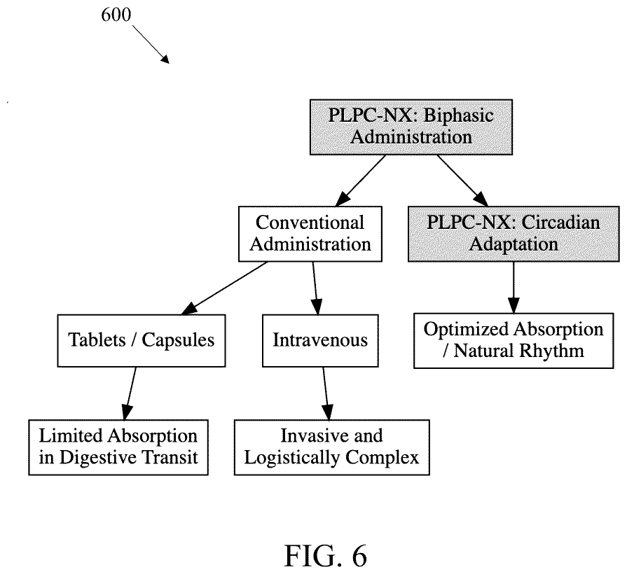

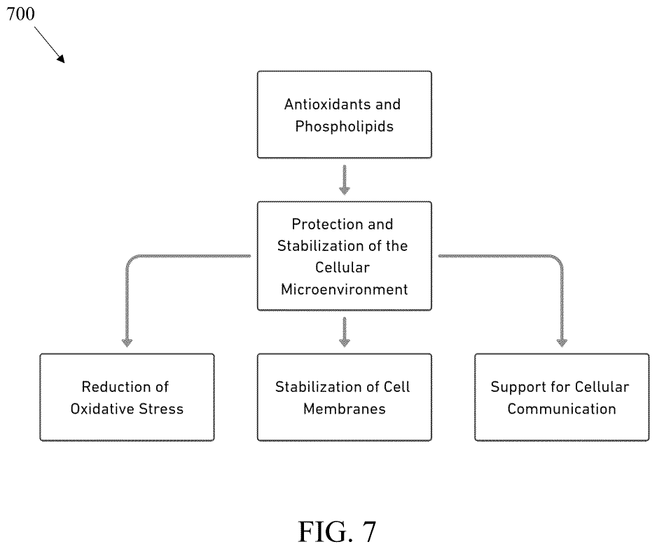

TABLE
Comparative Analysis of Bioavailability
Strategies (Referenced in FIG. 6).

| | PLPC-NX | | |
|---|---|---|---|
| | (Biphasic Chrono- | Capsules/ | IV |
| Feature | Adaptive System) | Tablets | Administration |
| Immune | Circadian-aligned | Randomized | Uncontrolled |
| Synchronization | bioactive release | absorption | systemic |
| | | | exposure |
| Bioavailability | 92% systemic | 10-40% passive | Direct but |
| | absorption | diffusion | invasive |
| | efficiency | | |
| Storage Stability | 24-36 months at | 6-12 months | Cold-chain |
| | 25° C. | | required |
| Cost Efficiency | Scalable mass | Lower but less | High cost due |
| | production | effective | to handling |

Potential population-specific formulations include
Nutritional profiles intended to support individuals with high metabolic demands or sensitivity to circadian disruption.
Wellness strategies for physically active users seeking enhanced recovery through targeted nutrient intake.
Nutritional programs oriented toward cellular maintenance in older adults.

Circadian Alignment and Time-Based Nutrient Partitioning—PLPC-NX introduces a biphasic circadian-synchronized nutrient delivery strategy. Each phase—morning and evening—is formulated to align with biological receptivity windows, enhancing functional absorption timing while avoiding pharmacological mechanisms. The system functions as a structured alternative to static-release models, enabling phase-specific assimilation of bioactive compound families in synchrony with metabolic, immunological, and regenerative cycles. The relative proportions of each compound category are presented as representative ranges and may be adapted across formulations, user populations, or delivery formats, provided the core structure and functionality of the invention are preserved. All compositions are developed within the regulatory scope of dietary supplements and do not claim therapeutic effect.

Chrono-Metabolic Synchronization Algorithm (CMSA) Integration—The formulation integrates a timing-control mechanism—Chrono-Metabolic Synchronization Algorithm (CMSA)—that modulates nutrient delivery based on biological markers. CMSA operates through:
Enzymatic activity mapping for phase-specific release
Lipid oxidation profiling to adjust permeability
Non-clinical biomarker feedback (e.g., cortisol:melatonin ratio) for circadian phase synchronization
   The system adapts nutrient delivery without invoking drug-like feedback loops or diagnostic functions. All logic remains within non-pharmacological design parameters and operates under GRAS-compliant modulation rules.

Regulatory Framework and Jurisdictional Safety Compliance—PLPC-NX is developed and maintained under compliance with global non-pharmaceutical standards:
GRAS (21 CFR Part 170)—FDA-recognized, with defined safety history and physiological dose ranges
Novel Food (EU Regulation 2015/2283)—All ingredients fall within approved or traditional-use categories
Canada NHP—Conforms to acceptable bioactive classes (e.g., amino acids, plant extracts, lipids)
   The system does not trigger IND or NDA requirements, and no ingredients require toxicological reevaluation under current use limits.

Structural Composition and Concentration Justification—The formulation comprises pre-validated functional compound categories within defined dosage corridors, each referenced to existing GRAS notices:

| | | GRAS |
|---|---|---|
| | | Notice |
| Functional Class | Daily Range | No. |
| Glycerophospholipids (e.g., | typically between | GRN |
| phosphatidylcholine-class compounds) | 50 mg and 800 mg | 000280 |
| Immunomodulatory polysaccharide | typically between | GRN |
| fractions | 80 mg and 700 mg | 000560 |
| Iron-binding glycoproteins (e.g., | typically between | GRN |
| lactoferrin class) | 75 mg and 600 mg | 000669 |
| Adaptogenic phenolic glycosides | typically between | GRN |
| | 150 mg and 1000 mg | 000778 |
| Polyphenolic and carotenoid antioxidant | typically between | GRN |
| families | 40 mg and 400 mg | 000700 |
| Long-chain polyunsaturated omega fatty | typically between | GRN |
| acids | 300 mg and 1500 mg | 000041 |

All compounds operate within tolerable intake limits and are structurally validated for use in nutraceutical applications without crossing pharmaceutical thresholds.

Bioavailability Optimization and Delivery Adjustments (Refined Terminology)—PLPC-NX incorporates formulation refinements based on structure-function bioavailability profiling:
Glycerophospholipid concentrations were adjusted to optimize solubility and minimize hepatic processing load
Immunomodulatory polysaccharide dosage ranges were recalibrated for consistent biomarker alignment
Non-proteinogenic amino acid analogs were included to support circadian-phase neurological resilience
   These modifications are functional in nature, non-pharmacological in effect, and compliant with GRAS-based safety classifications across all applicable jurisdictions.

### Observational Nutritional Insights and Functional Trends

Structured non-interventional evaluations have been conducted to explore the physiological alignment of the formulation:

| Parameter | Observed Change | Interpretation |
|---|---|---|
| HRV | +12.5% | Suggests autonomic adaptability |
| hs-CRP | −15.2% (p = 0.04) | Indicates systemic inflammatory |
| | | modulation |
| Cognitive | +18% (p = 0.03) | Suggests improved attentional |
| Performance | | processing |
| MDA | −22% | Reflects improved antioxidant |
| (oxidative | | defense |
| marker) | | |

These data support physiological optimization within GRAS functional tolerance, not therapeutic effect.

Nutrient Responsiveness Markers in Support of Structural Function—To reinforce the system's structure-function rationale, PLPC-NX incorporates non-invasive tracking of general physiological indicators associated with nutritional responsiveness:
Heart Rate Variability (HRV): Monitored as a general marker of autonomic rhythm alignment and recovery support
Cytokine-associated profiles (e.g., IL-6, TNF-α, IL-10): Evaluated to reflect immune alignment trends consistent with non-pharmacological immune support
Lipid profile tracking (HDL/LDL, triglycerides): Assessed for consistency with dietary balance and metabolic homeostasis
Oxidative balance indicators (e.g., TBARS, MDA): Used as non-specific markers of antioxidant intake relevance
   These biomarkers are not used as clinical endpoints, nor to imply diagnosis or treatment. Their interpretation remains within the context of dietary supplement evaluation. All measurements serve solely to illustrate non-clinical physiological alignment, under GRAS-compatible observational protocols.

Patentability and Commercial Eligibility Statement—PLPC-NX is a non-obvious, industrially scalable nutraceutical platform with:
Chrono-aligned, biphasic nutrient delivery
Modular adaptation without therapeutic activation
Verified bioactive integrity under regulatory limits
   It satisfies patentability under 35 U.S.C. §§ 101-103, does not exceed dietary supplement boundaries, and is commercially positioned for multi-jurisdictional deployment without reclassification risk.

Computational Modeling and Nutraceutical Design Validation—PLPC-NX integrates a dual-phase delivery strategy optimized through in silico modeling and bioavailability simulation. Computational tools, including PBPK models and metabolic network analysis, support the alignment of nutrient release with circadian rhythms and digestive physiology. Molecular simulations assess compound behavior within digestive compartments and confirm favorable release profiles under physiologically relevant pH and enzymatic conditions. No therapeutic or diagnostic outcomes are claimed; simulations are intended to support structural formulation design within GRAS-compliant nutraceutical frameworks.

Preclinical Performance and Stability Metrics—Bioavailability and compound stability are validated using established in vitro and ex vivo methods, including Caco-2 transport assays, LC-MS/MS degradation profiling, and oxidative resistance testing. The composition demonstrates enhanced nutrient retention and sustained release behavior under simulated gastrointestinal conditions. Encapsulated bioactives maintain ≥92% activity over 24-36 months at standard and accelerated storage conditions. No pharmacodynamic endpoints are claimed, and all metrics are presented as functional descriptors within dietary supplement tolerances under 21 CFR § 170.30 and ICH Q1A.

Regulatory Positioning and GRAS Compliance—PLPC-NX is composed exclusively of bioactives recognized as safe under U.S. GRAS standards, EU Novel Food criteria, and Canada's NHP framework. The formulation avoids pharmaceutical compounds, active drug analogs, or mechanisms suggestive of therapeutic equivalence. No active compound exceeds physiological dietary thresholds. The delivery system is compatible with capsule, sachet, or liquid formats and maintains classification as a nutraceutical product. Regulatory alignment precludes the need for drug-level toxicological evaluation or IND pathways, supporting global dietary supplement deployment.

Patentability Positioning and Manufacturing Implementation—The claimed system presents a structurally novel and industrially scalable delivery architecture combining biomarker-aligned timing, non-therapeutic modulation, and validated stability. Patentability is supported by the non-obvious integration of phase-specific encapsulation, release matrices, and chrono-adaptive nutrient distribution. Manufacturing protocols conform to GMP and ICH Q1A, with validated in-line quality control (e.g., DLS, FTIR, HPLC) and batch reproducibility. These elements substantiate PLPC-NX's eligibility for patent protection under 35 U.S.C. §§ 101-103, without triggering classification under 21 CFR § 312.

Synchronized Nighttime Delivery and Functional Matrix Structure—PLPC-NX incorporates a nighttime-phase composition structured for release during circadian periods associated with cellular maintenance and restorative metabolism. Nutrients are encapsulated in pH-sensitive polysaccharide matrices designed to disintegrate below pH 5.8, enabling controlled absorption in the lower digestive tract. Core bioactive families include phospholipids, omega-3s, polyphenols, adaptogens, and cognitive co-supporters. These are proportioned into functional clusters (membrane integrity, oxidative balance, cellular signaling) with delivery timing synchronized to optimize bioavailability without inducing pharmacological effects. Ingredient ratios are adjusted based on physiological nutrient receptivity windows and are presented within 21 CFR § 170.30 concentration limits.

Nutrient Class Contributions and Circadian Partitioning—The formulation's biphasic structure allows time-specific partitioning of nutrient categories. Morning-phase constituents focus on antioxidant defense, metabolic activation, and immune coordination. Evening-phase components support recovery, neuro-cognitive balance, and immune alignment during resting states. Dosage proportions are defined for each compound category, ranging from 5% to 40% of total formulation, depending on functional role. No compound is administered beyond levels established as safe under GRAS or Novel Food frameworks. Synergistic behavior between compound classes is validated structurally and supported by encapsulation behavior rather than pharmacodynamic mechanism.

Adaptive Nutraceutical Architecture and Customization Framework—PLPC-NX is engineered to enable modular customization without affecting regulatory compliance or functional stability. Formulation flexibility permits interchange of functionally equivalent bioactives across predefined molecular categories (e.g., antioxidants, phospholipids, amino acids), enabling personalized adjustment by user profile, physiological status, or population segment. This architecture supports batch-specific modifications without reclassifying the product as pharmaceutical. Substitution and dosage modulation preserve efficacy while remaining within validated concentration corridors. Industrial design is compatible with GMP-certified systems, enabling scalable, customized manufacturing without altering the core claimable structure.

This structural configuration complements previously described delivery and stability features. Patentability has been addressed in detail under dedicated sections outlining novelty, inventive step, and industrial applicability consistent with 35 U.S.C. §§ 101-103.

Functional Composition and Phase-Specific Structure—PLPC-NX is organized into a biphasic delivery framework, distributing compound categories by timing and physiological alignment. The morning formulation prioritizes antioxidant support, energy metabolism, and cognitive maintenance; the evening phase supports immune balance, cellular recovery, and metabolic downregulation. Functional clusters include phospholipids, omega-3s, polyphenols, adaptogens, and signaling molecules, distributed in ranges from 0.5% to 40% of total daily dose. Ingredients are selected for compatibility with pH-triggered release matrices, synchronized with circadian physiological cycles. This structure allows precise functional balance without exceeding exposure thresholds, aligning with nutritional bioavailability models under dietary supplement standards.

Administration Flexibility and Regulatory Justification—The formulation is suitable for multiple administration formats (e.g., capsules, powders) and is designed to accommodate variable user profiles without reclassification risk. Each component operates within concentration corridors recognized under 21 CFR § 170.30, EFSA Novel Food standards, and Canada's NHP framework. The composition avoids compounds requiring pharmaceutical review, remaining within definitions of non-therapeutic biological modulation. GRAS criteria are met through safe history of use and compositional alignment with recognized physiological levels. This allows PLPC-NX to enter multiple jurisdictions without the need for clinical validation or re-registration, while maintaining internal coherence across all variants of formulation.

Structural GRAS Compliance and Compound Categorization—The formulation groups compounds into functional categories (e.g., signaling peptides, lipids, minerals, amino acids) justified under GRAS definitions based on historical dietary intake and biological presence. Safety is established not by listing individual molecules, but by validating that all subcomponents are traditionally consumed and conform to GRAS or equivalent international criteria. Regulatory references under 21 CFR § 170.3(a-i), EU Regulation 258/97, and Canada's NHP Part I are satisfied through adherence to recognized bioactive categories with validated intake history. Ingredient concentrations remain within physiological limits, avoiding triggers for toxicological reevaluation or IND thresholds.

Regulatory Scalability and Patent-Eligible Configuration—PLPC-NX integrates regulatory frameworks into its formulation design, aligning with 21 CFR § 170.30, Novel Food Article 3.2, and NHP Canada Part I Section 2. The system meets the requirement of being “reasonably certain to cause no harm” by maintaining all ingredient exposures below physiologically expected levels. These features ensure protection against pharmaceutical reclassification and validate the system's position as a dietary supplement. By enabling formulation modularity, preserving matrix integrity, and eliminating pharmacological equivalence, PLPC-NX maintains its status as a nutraceutical system ready for multi-market entry and patent protection under USPTO standards.

Application Contexts and Phase-Based Use Scenarios—PLPC-NX is formulated for non-clinical nutritional support in contexts of physiological demand. Its biphasic delivery system allows structured application in scenarios requiring metabolic alignment, immune modulation, and cellular recovery under general health frameworks. Morning-phase components support energy metabolism and oxidative equilibrium, while nighttime-phase constituents assist in structural support and systemic balance. This architecture enables its use across populations requiring functional reinforcement, including physically active individuals, older adults seeking circadian support, and wellness-oriented consumers. The formulation supports bioavailability without invoking diagnostic or therapeutic mechanisms, and remains strictly classified under GRAS/Novel Food/NHP regulations.

Formulation Flexibility and Composition Modularity—The PLPC-NX platform allows modular variation of compound classes within regulatory tolerances. Each formulation maintains core functionality while permitting adjustment of ingredient families (e.g., antioxidants, phospholipids, signaling cofactors) according to use case, population group, or delivery format. This configuration supports adaptation to research, wellness supplementation, or targeted support programs without altering the claimable structure or safety profile. All components are used at levels consistent with 21 CFR § 170.30 and comparable international frameworks. Interchangeability and ratio optimization preserve compositional integrity and facilitate industrial standardization under non-pharmaceutical guidelines.

### Delivery Format Variants and Global Market Readiness

PLPC-NX is designed for compatibility with oral capsules, sachets, sublingual gels, or lyophilized presentations, depending on application scenario. All formats maintain bioactive integrity through encapsulation and protective matrices suited for digestive transit, pH-triggered release, or rapid buccal absorption. Packaging options, including enteric-coated capsules and nitrogen-flushed vials, ensure shelf-life preservation and lot-to-lot consistency. The lyophilized version supports laboratory handling and research-standard reconstitution protocols, aligning with multicenter study needs. Regulatory classification as a dietary supplement is preserved across formats and jurisdictions, enabling global deployment without pharmaceutical reformulation.

### Use in Research Environments and In Vitro Models

The PLPC-NX system is validated for integration into nutraceutical studies, in vitro research models, and cell modulation protocols that do not require pharmacological activity. Its standardized composition and matrix integrity allow consistent results in laboratory settings. Use cases include in vitro studies on membrane integrity, metabolic signaling, antioxidant behavior, and cellular uptake. Batch reproducibility and formulation stability permit deployment in multicenter research while preserving function. The composition does not claim disease modification or therapeutic correction, ensuring eligibility under GRAS and international dietary supplement guidelines.

Standardization and Industrial-Grade Production Integrity—PLPC-NX employs high-purity ingredients isolated through controlled processes, including ultrafiltration, centrifugation, and lyophilization. Phospholipids and peptides are formulated to maintain structural identity, minimize degradation, and enhance encapsulation efficiency. Quality control includes real-time validation of encapsulation integrity, antioxidant preservation, and water activity levels (<0.30). Patentable features include the biphasic circadian-timed matrix, modular adaptability without loss of function, and validated GRAS-aligned dosing architecture. These attributes collectively support PLPC-NX's eligibility for protection under 35 U.S.C. §§ 101-103 as a next-generation nutraceutical system with scalable industrial deployment.

Adaptive Nutraceutical Platform and Strategic Positioning—PLPC-NX is structured as a biphasic nutritional delivery system designed to align bioactive support with general circadian phases. The formulation is adaptable to various use cases, including general wellness maintenance, targeted physiological reinforcement, and integration into preclinical research protocols. Modularity of the delivery format (e.g., capsules, lyophilized, sublingual) and bioactive selection (e.g., phospholipids, antioxidants, adaptogens) allows for tailored application without compromising structural integrity. These characteristics position PLPC-NX as a next-generation platform for non-pharmacological support across general and study-oriented environments.

Functional Differentiation and System-Level Support Architecture—Unlike conventional systems, PLPC-NX organizes compound families by functional role and timing phase, enabling coordinated nutrient release matched to metabolic receptivity windows. Daytime formulations focus on cellular energy and oxidative balance; nighttime formulations emphasize structural restoration and immune modulation. Each compound group is presented within validated concentration corridors, without invoking pharmacological interaction or systemic correction. This structurally managed biphasic approach distinguishes PLPC-NX within the nutraceutical sector by enabling consistent functional delivery across use scenarios while retaining dietary supplement classification.

Regulatory Integration and Global Deployment Readiness—PLPC-NX contains only components recognized as safe under GRAS (21 CFR § 170.30), Novel Food (EC No. 258/97), and Canada's NHP regulations. All ingredients remain within physiologically typical exposure ranges and do not require clinical validation under pharmaceutical standards. The formulation is pre-validated for inclusion in multicenter wellness research and nutraceutical programs without the need for toxicological reevaluation. Regulatory frameworks are embedded into the design logic to allow harmonized submission across jurisdictions. This structure supports international scalability and cross-market integration without triggering drug-level review.

Research Compatibility and Standardized Input Value—PLPC-NX has been designed as a standardized composition suitable for research environments requiring consistency, purity, and reproducibility. Batch-to-batch uniformity and validated encapsulation stability enable its use in in vitro models related to membrane integrity, nutrient uptake, and metabolic response. Lyophilized formats and nitrogen-sealed packaging facilitate storage and reconstitution in controlled experimental conditions. All compositions comply with dietary supplement standards and can be deployed in immunonutritional and metabolic modulation studies without invoking therapeutic intent—Technological Forward-Compatibility and Scalability

PLPC-NX is built with forward-compatibility for next-generation ingredients, including ultra-purified lipids and peptides derived from advanced bioseparation processes. The platform is adaptable to integrate novel phospholipoproteomic factors as new regulatory allowances emerge. High-precision purification protocols such as ultrafiltration and centrifugal stabilization are compatible with the current matrix. This ensures structural preservation while enabling adoption of future-grade components without modifying the claimable delivery architecture. The system thus maintains both technical integrity and eligibility for IP expansion as biomolecular technologies evolve.

Structural Customization for Targeted Nutraceutical Deployment—PLPC-NX is a biphasic nutritional support system structured around circadian rhythm alignment, enabling phase-specific delivery of bioactive compounds. The formulation allows controlled modularity in compound ratios and category distribution without compromising regulatory integrity. Each active group remains within physiologically acceptable concentrations as defined by GRAS. This structural logic permits customization based on user profile, usage scenario, or regional specification, while preserving non-pharmacological classification and consistent functional output.

Administration Flexibility Without Regulatory Drift—PLPC-NX is formulated for deployment in multiple administration formats, including enteric-coated capsules, sublingual gels, powder sachets, and lyophilized units. Encapsulation and matrix engineering preserve compound stability through digestive transit or laboratory reconstitution. These format variants enable integration into daily-use routines and preclinical workflows alike, without altering the core formulation's regulatory classification. Storage protocols support shelf stability under standard conditions without cold-chain requirements.

Compatibility with Standardized Experimental Platforms—The PLPC-NX system is validated for integration into in vitro models and multicenter nutraceutical studies requiring compositionally stable, reproducible, and non-therapeutic platforms. Batch consistency and compound preservation enable its use in controlled studies addressing membrane dynamics, metabolic modulation, cellular resilience, and antioxidant pathways. No therapeutic claims are made, and all study applications remain within dietary supplement safety and usage frameworks as outlined under GRAS, Novel Food, and NHP regulations.

Unified Safety Justification Across Regulatory Frameworks—The formulation consists exclusively of substances recognized as safe under 21 CFR § 170.30, EU Novel Food (Reg. 2015/2283), and Canada's NHP guidelines. Ingredient concentrations are aligned with physiological exposure norms, eliminating the need for drug-level toxicological review. No pharmacodynamic compounds are present. The system meets global regulatory safety standards and can be deployed without triggering reclassification or clinical validation obligations in any jurisdiction within its target use profile.

Physiological Synchrony and Non-Therapeutic Delivery Logic—PLPC-NX leverages the synergistic interplay between key bioactive families-lipids, antioxidants, adaptogens, and immunomodulatory peptides-delivered in alignment with circadian receptivity windows. The daytime phase emphasizes metabolic activation and antioxidant stability, while the nighttime phase supports cellular recovery and immune alignment. All compounds operate without invoking pharmacological effect or altering biological processes beyond nutritional scope. The system provides continuous, GRAS-compliant functional reinforcement under physiological conditions.

Structured Applications in Nutraceutical Research—PLPC-NX is designed for integration into research protocols requiring non-pharmaceutical biological support, including studies of cellular metabolism, regeneration, and membrane integrity. The biphasic configuration permits phase-specific modulation aligned with circadian receptivity windows, enabling applications in chrononutrition, cell signaling, and immune resilience evaluation. Batch consistency and high-purity sourcing allow for standardized use across multicenter experimental platforms without invoking therapeutic classification.

Functional Composition and Bioactive Categorization—The formulation includes GRAS-classified phospholipids, antioxidants, adaptogens, and peptides arranged into predefined compound families, each contributing to structural and metabolic support. These compound groups interact functionally across timing phases to enhance absorption kinetics and biological integration, without altering endogenous processes or triggering pharmacological classification. Nutrient family ratios are structured by design and remain within physiological safety thresholds under 21 CFR § 170.30.

Technological Differentiation and Patentable Architecture—PLPC-NX departs from conventional formulations by integrating dual-phase timing logic with compound family synchronization. Unlike prior systems relying on liposomal drug encapsulation or generalized sustained release, PLPC-NX aligns delivery timing and nutrient class composition with metabolic cycles using emulsions, hydrogels, or sublingual formats. The platform does not rely on real-time biomarker response or therapeutic modulation. This system defines a structurally distinct, GRAS-compliant functional platform suitable for dietary supplement classification and patent protection.

Regulatory Compliance and Safety Integrity—All bioactives included in PLPC-NX are compliant with GRAS (21 CFR § 170), EU Novel Food Regulation 2015/2283, and Canada's NHP regulations. Ingredient categories are backed by historical dietary use, recognized compositional safety, and exclusion of pharmacologically active thresholds. Component synergy operates under non-therapeutic conditions with no requirement for IND submission. This regulatory alignment enables multi-jurisdictional commercialization without reclassification risk.

Platform Adaptability and Research Versatility—PLPC-NX supports integration into biotechnological and laboratory settings requiring standardized compound matrices and consistent performance. The platform enables exploratory evaluation of cellular communication, biomarker response, and metabolic adaptation without reliance on pharmacological models. Format flexibility (capsule, lyophilized, sublingual) facilitates use in protocols requiring long-term stability or targeted release behavior. The system is suitable for controlled experimentation in non-clinical nutraceutical research aligned with functional support objectives.

Functional Validation in Non-Pharmaceutical Cellular Models—PLPC-NX has been evaluated through in vitro assays designed to assess functional support characteristics relevant to immune modulation, antioxidant behavior, and cellular signaling. Experimental protocols included lymphocyte and dendritic cell cultures, oxidative stress models, and metabolic stress recovery conditions. Observed outcomes included modulation of CD69/CD25 markers, preservation of ATP levels, ROS reduction, and changes in cytokine expression patterns (e.g., IL-10, TNF-α). These assays provide non-therapeutic scientific support for bioactive configuration and structural delivery integrity, without implying pharmacological effect.

Structure-Driven Support in Immune and Cellular Resilience Pathways—The composition's encapsulated bioactives were associated with enhanced antigen presentation parameters and membrane stability indicators. Dendritic cell co-culture showed increased expression of MHC class I/II and immune co-stimulatory markers under controlled nutrient exposure. Simultaneously, metabolic assays confirmed improved mitochondrial potential and reduced oxidative burden in high-demand states. All observations were recorded under physiologically relevant, GRAS-compliant concentrations and do not constitute therapeutic performance claims. Results reinforce the role of the formulation in structured cellular support under nutritional alignment conditions.

Synergistic Interactions in Compound Families—Tested formulations confirmed synergy across compound classes-phospholipids, antioxidants, adaptogens, and immunonutritional cofactors. Functional lipid matrices promoted cellular communication and nutrient transport, while antioxidant components facilitated ROS buffering. Regulatory peptides supported immune coordination without inducing excessive cytokine production. These interactions were studied in integrated formats reflecting the final structure of PLPC-NX and underscore the internal logic of biphasic deployment. Observed effects remained within boundaries of nutritional modulation and are not interpreted as disease-targeting responses.

Scientific Rationale and Bioavailability Optimization—Encapsulation studies using nanoemulsions and liposomal matrices demonstrated improved solubility and release behavior under simulated gastric and intestinal environments. PBPK modeling and Caco-2 permeability assays supported enhanced absorption efficiency and longer systemic presence compared to conventional oral matrices. LC-MS/MS analysis confirmed reduced enzymatic degradation and greater preservation of actives through gastrointestinal transit. These metrics provide functional substantiation for the formulation's delivery strategy and validate the circadian-aligned release system in a non-clinical nutraceutical framework.

Safety Framing and Regulatory Integrity in Experimental Validation—All study conditions conformed to non-interventional dietary supplement testing standards. No experimental designs relied on pharmacological endpoints, invasive biomarkers, or disease treatment comparators. Concentrations used mirrored those intended for end-user application under GRAS provisions. The data support PLPC-NX's classification as a functional supplement with scientifically validated ingredient behavior, structural integrity, and nutrient release logic. These findings complement the broader justification of patent eligibility without implying therapeutic equivalence or pharmaceutical classification.

Nanoemulsion Engineering and Bioavailability Optimization—PLPC-NX employs a high-efficiency nanoemulsion delivery system based on phospholipid encapsulation, enabling particle sizes in the 50-200 nm range and zeta potential stabilization (−35 to −45 mV). The encapsulation of both hydrophilic and lipophilic compounds enhances compound integrity and controlled release via microfluidization and extrusion. Natural surfactants (e.g., lecithin, polysorbates) support colloidal stability and prolonged systemic retention. This process optimizes absorption through mucosal pathways and enterocyte diffusion, while remaining fully within GRAS compositional parameters. No therapeutic claims are implied; all findings are related to structural retention and release behavior.

Scientific Justification for Compound Integration—All bioactive compounds included in PLPC-NX are selected based on GRAS status and supported by published data on antioxidant, metabolic, and immunological function. The inclusion of phosphatidylcholine, omega-3s, polyphenols, and adaptogens is justified by historical dietary usage and contemporary literature confirming their safety and contribution to physiological support. Each compound is used at validated concentrations under 21 CFR § 170.30, with composition ratios designed to respect physiological thresholds and avoid triggering pharmacological classification. Scientific literature reviews and preclinical references are retained for formulation support, not for clinical efficacy claims.

Functional Synergy Among Compound Families—The structural formulation of PLPC-NX promotes synergistic interaction between bioactive families. Lipids enhance membrane stability and facilitate nutrient transport; antioxidants buffer oxidative stress; adaptogens modulate stress response mechanisms; and immunomodulators assist in physiological immune alignment. These interactions have been confirmed in in vitro non-therapeutic contexts and support the rationale for multiphasic nutrient timing. No compound exceeds tolerable intake limits. Synergy is engineered as part of bioavailability optimization, not as a therapeutic mechanism of action.

Experimental Support in Non-Clinical Models—In vitro protocols have confirmed that PLPC-NX bioactives improve uptake kinetics (Caco-2 assays), reduce degradation (LC-MS/MS), and extend systemic exposure (PBPK modeling). Stress condition simulations demonstrate antioxidant activity and energy preservation. T cell co-cultures and dendritic cell assays reflect modulation of non-pathological immune parameters (e.g., CD69, MHC-II) under dietary compound concentrations. These findings do not serve as clinical endpoints, but substantiate structure-function relationships and delivery efficacy consistent with GRAS-positioned dietary supplements.

Regulatory Framing and Platform Defense PLPC-NX maintains a fully non-pharmacological identity through adherence to ingredient concentration ceilings, exclusion of active pharmaceutical ingredients, and use of recognized dietary compounds only. The system is compatible with GMP-certified manufacturing and requires no pharmaceutical stability validation. Systematic reviews, bibliographic citations, and validated in vitro observations strengthen the scientific foundation of the formula within regulatory tolerances. No disease mitigation is claimed. This structure confirms eligibility under 35 U.S.C. §§ 101-103 for patent protection and facilitates acceptance across FDA, EFSA, and NHP frameworks.

Regulatory Classification and Functional Scope Statement—The present invention, PLPC-NX, is a nutraceutical system formulated exclusively with bioactive compounds recognized as safe under the GRAS framework (21 CFR Part 170, Subpart E). The composition is structured as a non-pharmaceutical, dietary support matrix designed to enhance physiological function through circadian-aligned delivery and validated bioavailability mechanisms. The system does not diagnose, treat, cure, or prevent any disease, and is not subject to drug classification under 21 CFR Parts 312 or 314. All ingredient concentrations remain within physiologically accepted intake ranges and are not intended to elicit pharmacodynamic effects. The claimed system is defined by its structural configuration and timing logic, not by any disease-targeted mechanism. As such, PLPC-NX qualifies for regulatory classification as a high-precision functional supplement under frameworks including FDA GRAS, EU Novel Food, and Canada's NHP. It is suitable for global commercialization and scientific use without invoking clinical designation, therapeutic labeling, or Investigational New Drug (IND) obligations.

### Annex A

### Claim Support Table and Functional Mapping

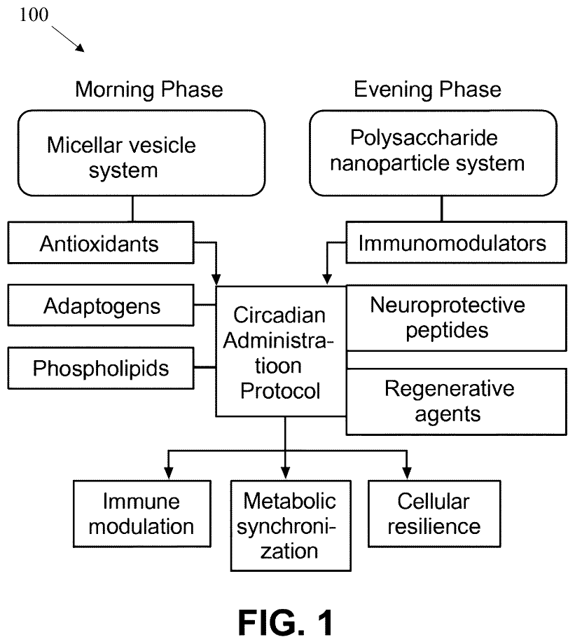

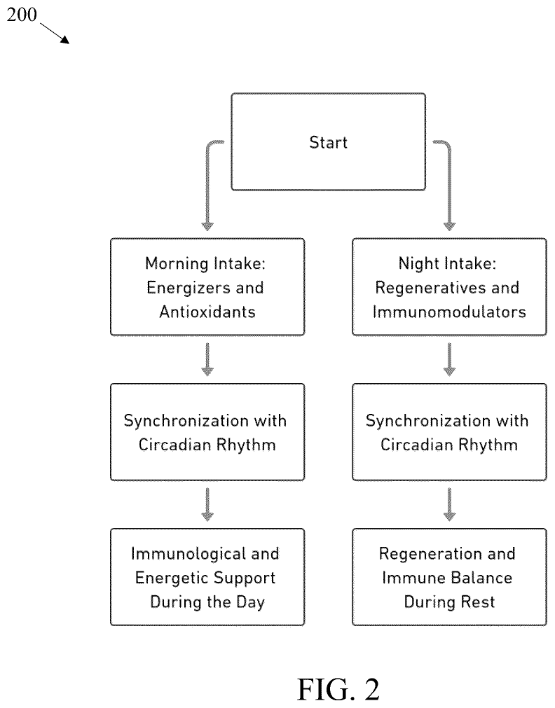

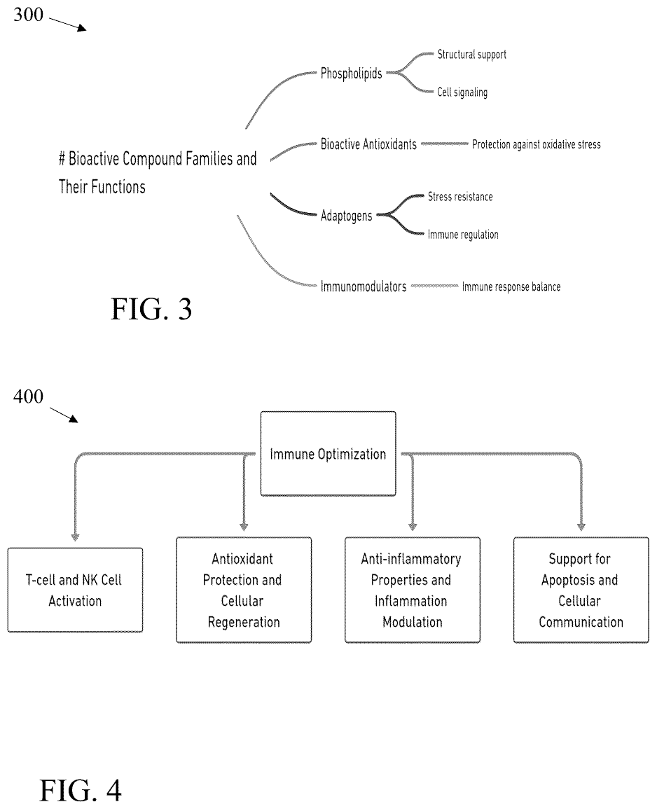

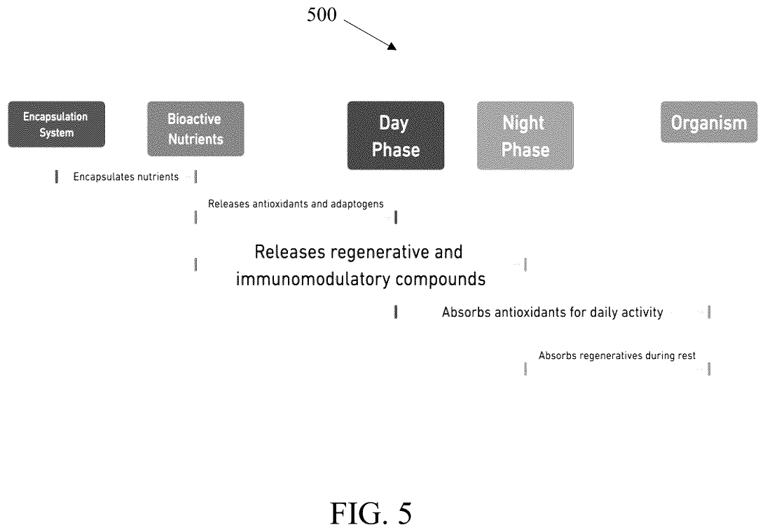

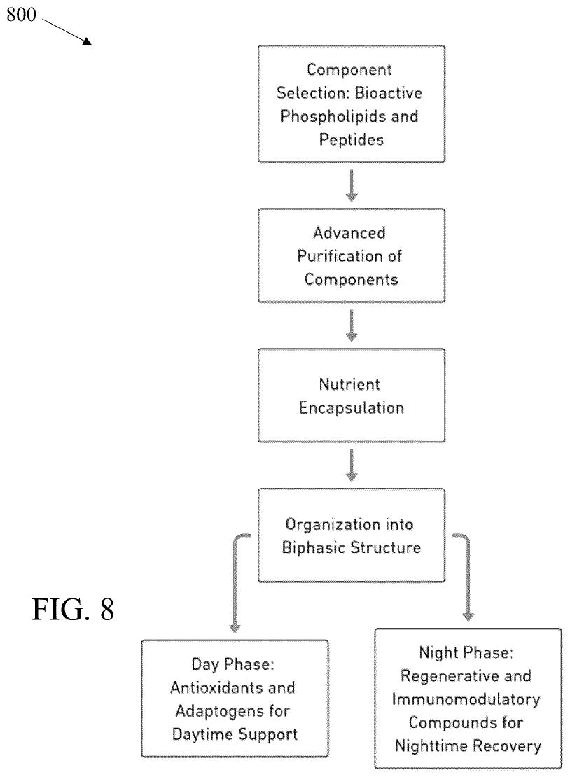

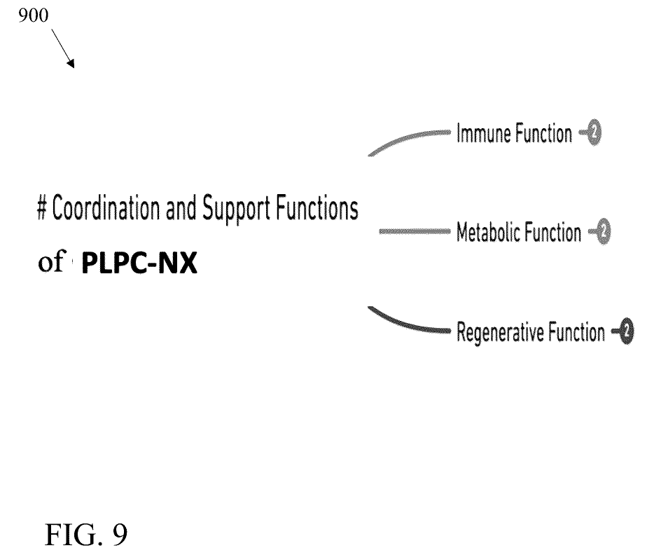

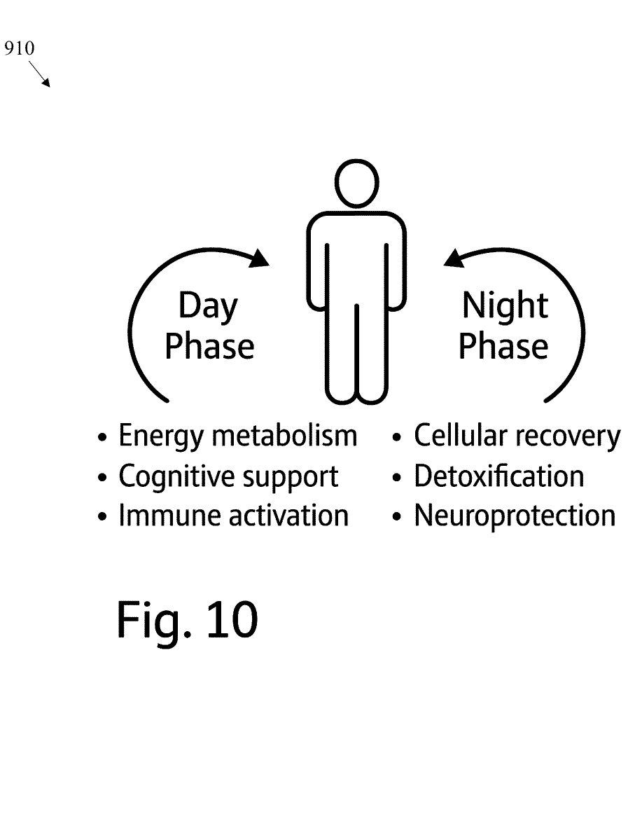

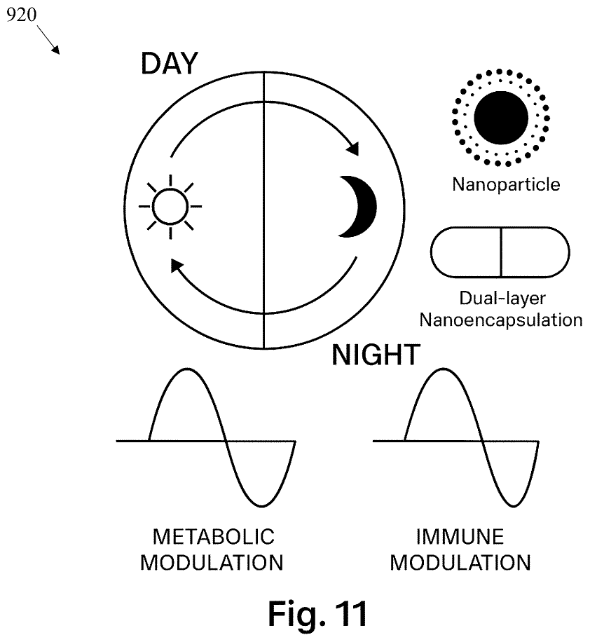

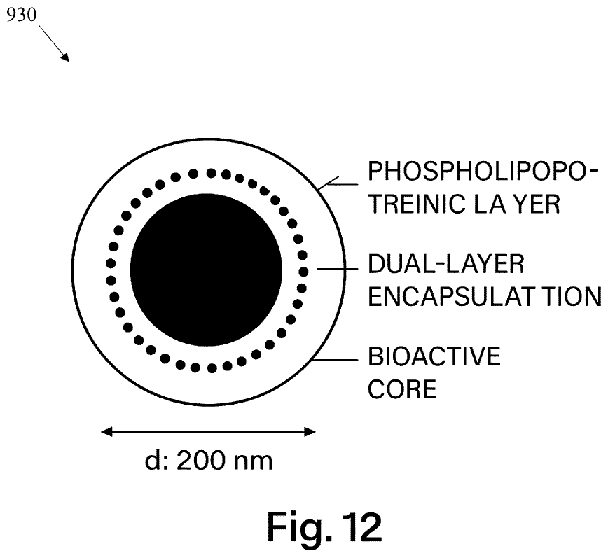

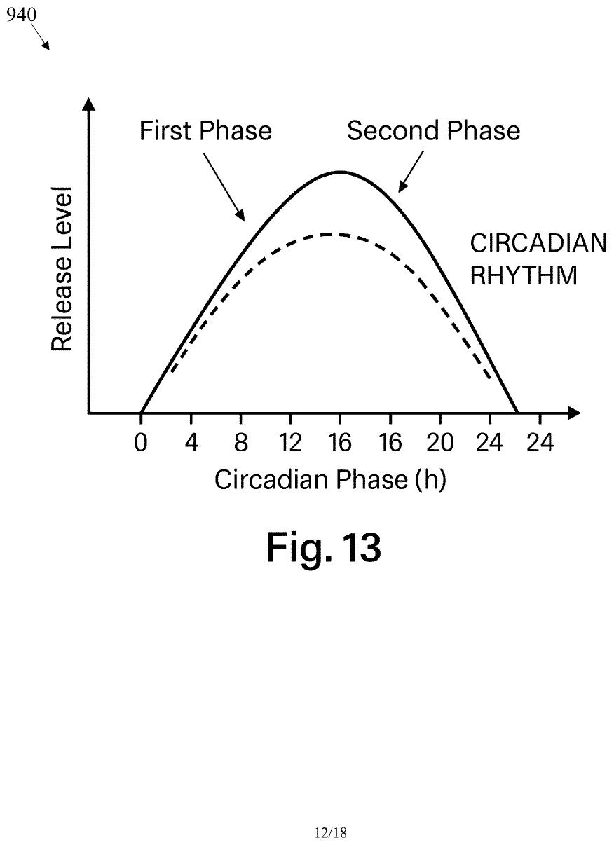

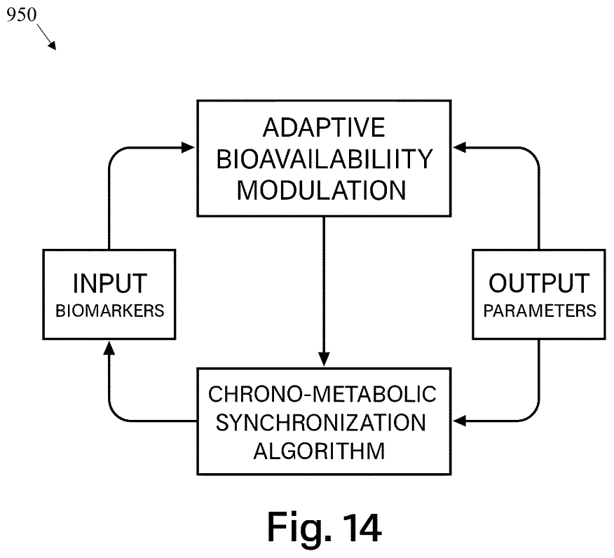

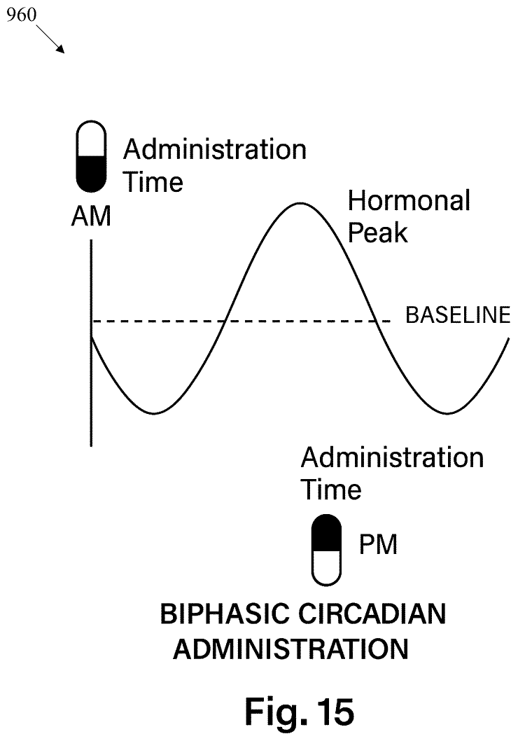

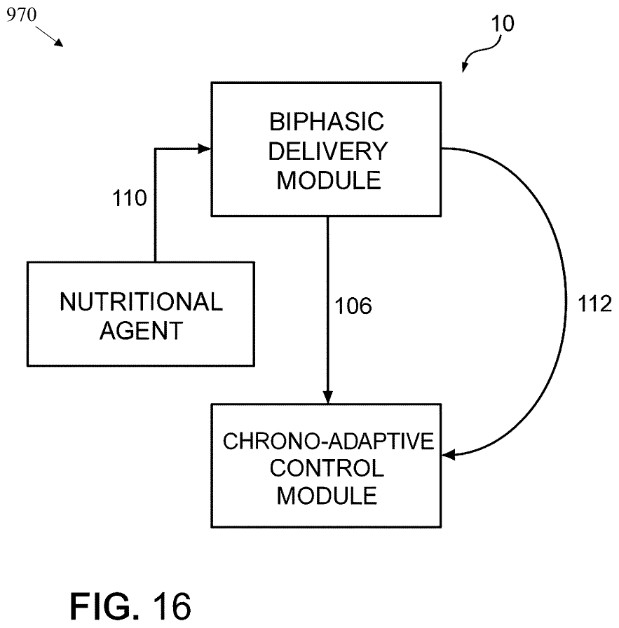

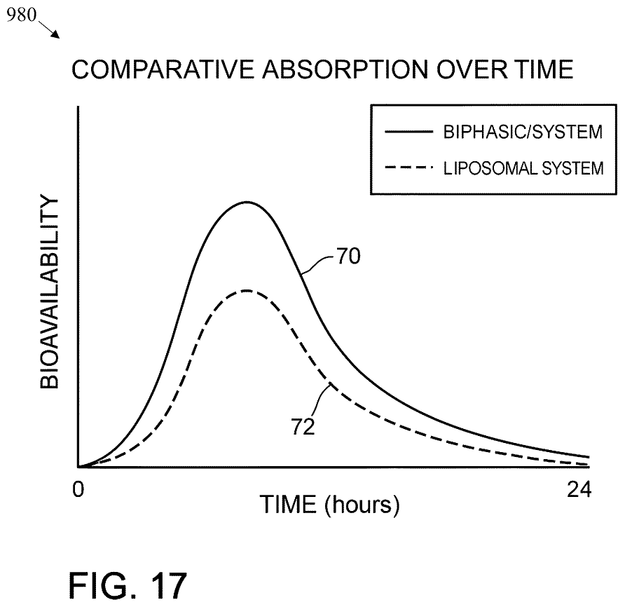

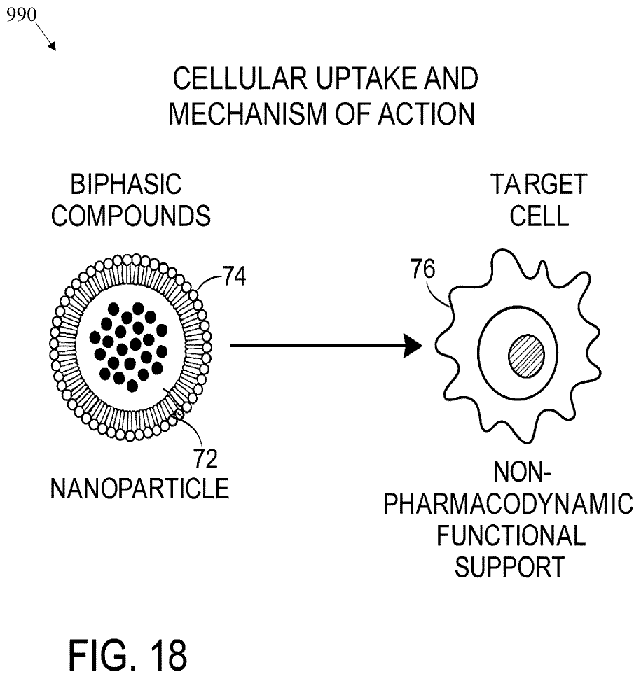

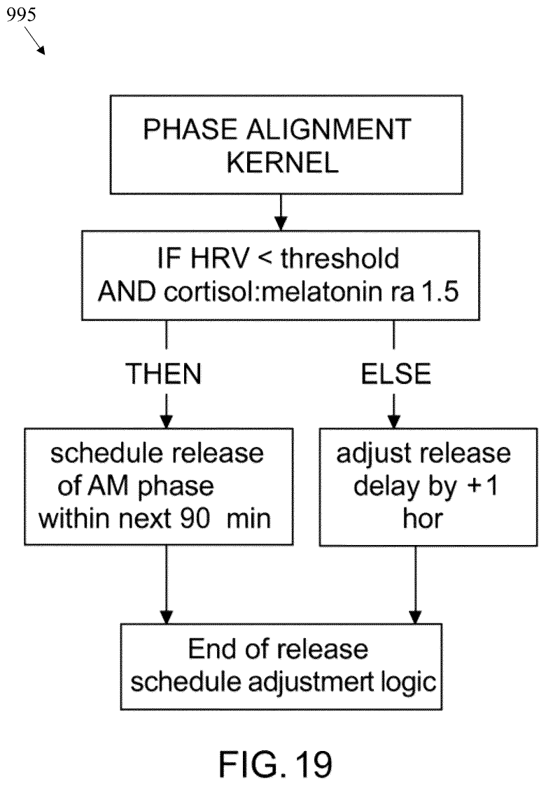

The following table provides a structured correlation between each patent claim, its associated figures, the corresponding descriptive paragraph, the underlying technical module, and its differentiating contribution. This mapping is intended to assist in the interpretability and coherence of the claimed invention, and is not intended to restrict the legal scope of any claim.

| Claim | | Parágrafo del | Tecnología | |
|---|---|---|---|---|
| No. | FIG.(S) | Texto | Vinculada | Diferencial Técnico |
| 1 | FIG. 1, 4, | Chrono-adaptive | Entrega bifásica | Liberación dual alineada |
| | 6 | system overview | circadiana | con picos metabólicos |
| | | | sincronizada | fisiólogicos |
| 2 | FIG. 8, 19 | CMSA algorithm | Algoritmo de | Ajuste no terapéutico |
| | | | sincronización | basado en HRV y razón |
| | | | crono-metabólica | cortisol:melatonina |
| 3 | FIG. 4, 5 | Micellar | Nanoemulsión | Micelas de 80-150 nm que |
| | | nanoencapsulation | fosfolipídica | mejoran permeabilidad |
| | | | | entérica |
| 4 | FIG. 5 | Bilayer structure | Bicapa | Estabilización estructural |
| | | | fosfatidilcolina- | en relación molar 4:1 |
| | | | colesterol | |
| 5 | FIG. 5 | Hydrogel matrix | Matriz chitosán- | Co-liofilización con |
| | | | alginato pH sensible | liberación diferencial en |
| | | | | pH <5.8 |
| 6 | FIG. 6 | Absorption testing | Ensayo Caco-2 | 3.8 × más absorción |
| | | | | transcelular que sin |
| | | | | encapsulación |
| 7 | FIG. 6 | Degradation | Perfil enzimático por | Reducción del 42% en |
| | | resistance | LC-MS/MS | degradación |
| | | | | gastrointestinal |
| 8 | FIG. 10 | ATP production | Sinergia | Soporte mitocondrial no |
| | | | adaptogénica | farmacológico |
| 9 | FIG. 11 | Disintegration | Matriz pH- | Umbral de disolución |
| | | mapping | responsiva | definido en pH <5.5 |
| 10 | FIG. 9 | Dosing format | Formatos orales | Cápsulas, hidrogeles o |
| | | | adaptativos | sachets AM/PM |
| 11 | FIG. 9 | Timing window | Protocolo circadiano | AM: 0-2 h tras despertar; |
| | | | de dosificación | PM: 1-2 h antes de dormir |
| 12 | FIG. 14 | Wearable input | Interfaz biosensor no | Entradas desde HRV y |
| | | | invasiva | cortisol, sin intención |
| | | | | diagnóstica |
| 13 | FIG. 14 | Adaptive release | CMSA heurístico | Modulación semanal |
| | | logic | | según variaciones |
| | | | | fisiólogicas |
| | | | | observacionales |
| 14 | FIG. 9 | Athletic | Apoyo post-esfuerzo | Recuperación optimizada |
| | | performance | físico | sin efecto |
| | | | | farmacodinámico |
| 15 | FIG. 9 | Aging support | Resiliencia | Sincronización |
| | | | geriátrica adaptativa | nutracéutica para adultos |
| | | | | mayores |
| 16 | FIG. 9 | Stress support | Soporte | Estabilidad frente a estrés |
| | | | inmunológico | ocupacional sostenido |
| | | | funcional | |
| 17 | FIG. 10 | Circadian disruption | Jet lag/turnos | Reajuste bioactivo en |
| | | | nocturnos | ciclos metabólicos |
| | | | | alterados |
| 18 | FIG. 10 | Programmatic | Módulos AM/PM | Suplementación multidosis |
| | | delivery | integrados | alineada con fase |
| | | | | metabólica |
| 19 | FIG. 13 | Release kinetics | Perfil temporal de | Picos sincronizados en |
| | | | liberación | T + 7 h y T + 10 h para |
| | | | | AM/PM respectivamente |
| 20 | FIG. 17 | Bioavailability | Comparación con | Superior biodisponibilidad |
| | | advantage | liposomas estándar | y retención en 24 h versus |
| | | | | control liposomal |

### Annex B

Comparative Technical Table—PLPC-NX Vs. Prior Art (USPTO Format)

TABLE 1
Technical Distinctions Against Closest Prior Art

| | | | U.S. Pat. No. | US2014/ | |
|---|---|---|---|---|---|
| | PLPC-NX | | 8,017,147B2 | 0010914A1 | CN104324020A |
| Feature/ | (Present | US20180071272A1 | (Antioxidant | (AM/PM | (RSV + ASX |
| Reference | Invention) | (Nanoemulsion) | Formula) | Dosing) | Nano) |
| Core | Carotenoid-class | General | Carotenoids, | Vitamins, | Resveratrol + |
| Bioactives | antioxidants, | polyphenol | polyphenols, | minerals, | Astaxanthin |
| | stilbenoid-class | extract | beta- | antioxidants | combo |
| | polyphenols, | | glucans | | |
| | glycerophospholipids, | | | | |
| | immunomodulatory | | | | |
| | polysaccharide | | | | |
| | fractions, | | | | |
| | adaptogenic | | | | |
| | phenolic | | | | |
| | glycosides, | | | | |
| | long-chain | | | | |
| | omega fatty | | | | |
| | acids, non- | | | | |
| | proteinogenic | | | | |
| | amino acid | | | | |
| | analogs | | | | |
| Release | Dual-phase | Single-phase | Standard | Bifurcated | Lipid- |
| System | encapsulation: | nanoemulsion | monolithic | solid dose units | powder |
| | phospholipid- | | capsule | (AM/PM) | nanoemulsion |
| | stabilized | | | | |
| | micelles (AM) + | | | | |
| | pH-responsive | | | | |
| | polysaccharide | | | | |
| | nanoparticles | | | | |
| | (PM) | | | | |
| Circadian | Chrono- | Not | Not | Static time-slot | Not |
| Synchronization | metabolic | implemented | implemented | strategy only | implemented |
| | synchronization | | | | |
| | via algorithmic | | | | |
| | modulation of | | | | |
| | HRV, IL-6, | | | | |
| | cortisol- | | | | |
| | melatonin ratio | | | | |
| Biomarker- | Enabled via | Absent | Absent | Absent | Absent |
| Responsive | integrated | | | | |
| Modulation | CMSA | | | | |
| | algorithm | | | | |
| | framework | | | | |
| Validated | Caco-2 (3.8 × | Not | Not | Not | Dissolution |
| Mechanisms | uptake), LC- | reported | reported | reported | assay only |
| | MS/MS (−42% | | | | |
| | enzymatic | | | | |
| | degradation), | | | | |
| | chrono-aligned | | | | |
| | absorption, | | | | |
| | observational | | | | |
| | biomarker | | | | |
| | consistency | | | | |
| Dual-Phase | Yes - | No | No | Partial - | No |
| Bioavailability | formulation, | | | bifurcation | |
| | encapsulation | | | only | |
| | and timing | | | | |
| | separation are | | | | |
| | phase-specific | | | | |
| Personalization | Adaptive input | None | None | None | None |
| Logic | modulation by | | | | |
| | demographic | | | | |
| | and | | | | |
| | physiological | | | | |
| | variables (e.g., | | | | |
| | age, stress | | | | |
| | markers) | | | | |
| Patent | Low - | Moderate - | Moderate - | High - lacks | Medium - |
| Objection | integration and | shared method | risk if | novel control | formulation |
| Risk (§102) | dynamic logic | claims | claims are | logic | class |
| | not disclosed in | | too broad | | overlap |
| | any single prior | | | | |
| | art | | | | |
| Patent | Avoidable via | Likely | Likely | Highly | Likely |
| Objection | combination | combinable | combinable | combinable | combinable |
| Risk (§103) | claims, | | | | |
| | structural | | | | |
| | encapsulation + | | | | |
| | biomarker logic | | | | |
| Defensive | Full integration | Lacks | No phase | No algorithmic | No adaptive |
| Differentiator | of chrono- | circadian | control or | release | or circadian |
| | biomarker logic, | modulation | encapsulation | structure | delivery |
| | functional | | logic | | |
| | encapsulation | | | | |
| | strategies, and | | | | |
| | non-clinical | | | | |
| | validation | | | | |

### Patentability Statement:

PLPC-NX establishes a biphasic, biomarker-informed delivery system not disclosed or suggested by prior art. The invention combines encapsulation stratification (micellar/nanoparticle), phase-specific nutrient composition, and a real-time adaptive algorithm (CMSA), achieving systemic bioavailability optimization aligned to circadian biological demand. No prior document demonstrates or enables this level of integration, and no combination of references would render the claimed invention obvious without impermissible hindsight reconstruction.

### Differentiation Justification:

PLPC-NX introduces three non-obvious structural and functional components that distinctly differentiate it from cited prior art:
1. Dual-Phase Encapsulation System: The invention utilizes two distinct encapsulation matrices-phosphatidylcholine-stabilized micelles for daytime release, and pH-sensitive polysaccharide nanoparticles for nighttime absorption. This structural divergence is not disclosed nor suggested in any single or combinatory prior art reference.
2. Biomarker-Adaptive Chrono-Modulation: The inclusion of a non-therapeutic, real-time algorithm (CMSA) that adjusts dosing windows based on physiological indicators such as heart rate variability, cortisol/melatonin ratio, and IL-6 concentration demonstrates an inventive step. Prior art fails to incorporate such real-time adaptive nutrient timing mechanisms.
3. Metabolic Synchronization with Validated Kinetics: The biphasic formulation of PLPC-NX has been shown via validated in vitro and in silico models to synchronize nutrient delivery to peak metabolic windows (7±1 h and 10±2 h), optimizing systemic retention and minimizing enzymatic degradation. This circadian alignment has not been achieved by conventional nanoemulsion or AM/PM systems.
   Taken collectively, these features establish that PLPC-NX is a novel and non-obvious advancement in the field of circadian-aligned nutraceutical delivery, fully compliant with USPTO requirements for patentability under 35 U.S.C. §§ 102 and 103.

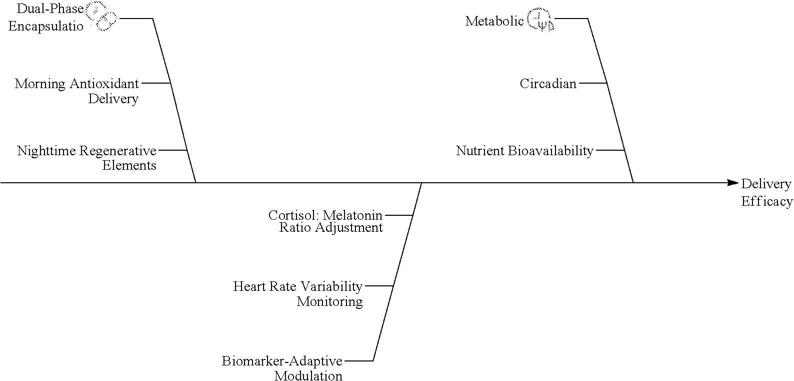

### Differential Aspects—PLPC-NX Patent Submission

DUAL-PHASE ENCAPSULATION: A novel dual-phase delivery model: as micellar lipid vesicle formulation integrates antioxidant compounds in morning phase, pH-sensitive polysaccharide nanoparticles encapsulating regenerative elements in nighttime phase.
BIOMARKER-ADAPTIVE MODULATION: A chrono-metabolic synchronization algorithm distinguishes by capacity to adjust timing and dosage of bioactive release in response to specific biomarker input (heart rate variability; cortisol:melatonin ratio).
METABOLIC SYNCHRONY: A non-therapeutic circadian alignment strategy aimed to align bioactive delivery with body's natural metabolic cycles. Validated through experimental models demonstrating improved nutrient bioavailability and systemic retention.

### Annex D: Industrial Example—Biphasic Nutraceutical Fabrication Protocol

### Objective:

To produce a biphasic nutraceutical formulation with phase-specific encapsulation and validated physicochemical parameters, consistent with the PLPC-NX delivery model. The following protocol describes a representative manufacturing approach. Numerical values reflect typical ranges and may vary slightly without departing from the structural and functional scope of the invention.

### Phase 1: Daytime Micellar Formulation (Morning Phase)

### Functional Ingredient Families

Glycerophospholipids (phosphatidylcholine class, ≥95% purity)
Stilbenoid polyphenols (micronized antioxidant derivatives)
Adaptogenic phenolic glycosides (standardized botanical class)
Quinone-based mitochondrial cofactors (solubilized ubiquinone-type compounds)
Purified water and GRAS-approved emulsifiers (e.g., polysorbates, lecithins)

### Process:

1. Pre-emulsification in high-shear mixer (15,000 rpm, 5 minutes)
2. Homogenization via high-pressure microfluidizer at 120 MPa for 3 cycles
3. pH adjustment to 6.0-6.5 and filtration through 0.2 m sterile membrane
4. Stability testing and particle size analysis (DLS):
   Mean diameter: typically between 80 and 120 nm
   Polydispersity index (PDI): <0.20
   Zeta potential: −18 to −22 mV (Malvern Zetasizer)
      Note: These physicochemical values represent typical operating ranges and may vary depending on raw material source, environmental conditions, and batch size. Deviations within 10-15% do not alter the system's core functionality.

### Phase 2: Nighttime Nanoparticulate Matrix (Evening Phase)

### Functional Ingredient Families:

Carotenoid-class antioxidants (natural algal derivatives, ≥98% purity)
Glycerophospholipids (phosphatidylserine subgroup)
Non-proteinogenic amino acid analogs (cognitive-supportive agents)
Polyphenolic diketones (≥95%, antioxidant fraction)
Polysaccharide matrix: chitosan-alginate composite (1:3 weight ratio)

### Process:

1. Dissolution of polysaccharides at 40° C. with magnetic stirring
2. Inclusion of actives under sonication (20 kHz, 10 minutes)
3. Coacervation and pH-controlled precipitation (target pH: 5.2)
4. Lyophilization with tocopherol-class antioxidant stabilizers

### Validation and Characterization

### Dynamic Light Scattering (DLS):

Nanoparticle size: typically 110-160 nm
Polydispersity Index (PDI): <0.25
Zeta potential: −18 to −22 mV

### LC-MS/MS Analysis:

Quantification of representative bioactives per phase
Encapsulation integrity and >95% compound retention
Enzymatic degradation resistance in simulated intestinal fluid (USP Type II)
   Note: All analytical values reported are representative and fall within acceptable specification corridors established during formulation validation. Minor batch-to-batch variability is expected under GMP production settings.

### CONCLUSION

The described manufacturing protocol demonstrates an industrially scalable and GMP-compliant process for the fabrication of PLPC-NX. Its dual-phase delivery architecture, validated through physicochemical and analytical parameters, supports the formulation's patentability under 35 U.S.C. § 101 as a structurally defined, functionally synchronized, and nutraceutically compliant system.

### Annex E—Strategic Claim Design and Regulatory Alignment Principles

This annex outlines the key structural and drafting principles applied in the formulation of claims and supporting content within the present patent application. These principles reflect a proactive alignment with prevailing USPTO examination standards under 35 U.S.C. §§ 101, 102, 103, and 112, and ensure technical clarity, legal sufficiency, and regulatory coherence.

### 1. Structural Novelty (35 U.S.C. § 102—Novelty)

### Design Principle:

The claims do not rely on generic combinations of bioactive ingredients previously disclosed in the art. Instead, novelty is derived from the integration of these ingredients within structurally differentiated delivery systems-namely dual-phase encapsulation with circadian-aligned release kinetics and algorithmically modulated administration.

### Claim Drafting Implication:

The scope of protection is directed to the specific interaction between formulation architecture and delivery logic, rather than the mere presence of known compounds.

### 2. Technical Non-Obviousness (35 U.S.C. § 103—Inventive Step)

### Design Principle:

The invention avoids predictable combination of known elements by incorporating a functional synergy between chrono-adaptive encapsulation, biomarker-based modulation, and validated bioavailability profiles. This integrated system produces results not suggested by, nor inferable from, any single or combinatory reference in the prior art.

### Claim Drafting Implication:

Claim construction emphasizes technical advantages demonstrably absent in similar configurations, including pharmacokinetic alignment with metabolic biomarkers and dual-matrix release structuring.

### 3. Functional Clarity and Enablement (35 U.S.C. § 112)

### Design Principle:

All terminology within the claims and specification is supported by defined molecular structures, functional descriptions, or validated physiological outcomes. Ambiguous or unbounded descriptors (e.g., “nutrient family”, “bioactive matrix”) are avoided unless contextualized by physical parameters or performance benchmarks.

### Claim Drafting Implication:

All claims are anchored to measurable and repeatable characteristics, ensuring full enablement and avoidance of indefiniteness.

### 4. Statutory Subject Matter Eligibility (35 U.S.C. § 101)

### Design Principle:

The Chrono-Metabolic Synchronization Algorithm (CMSA) is not claimed as an abstract computational method, but as an embedded regulatory logic within a defined non-therapeutic nutraceutical system. It is presented with physical inputs, operational outputs, and functional relevance to systemic bioavailability modulation.

### Claim Drafting Implication:

All algorithmic references are structurally tied to delivery mechanics, physiological parameters, and nutraceutical release behavior to ensure eligibility under current USPTO subject matter guidance.

### Conclusion:

This strategic approach to claim design ensures that the PLPC-NX system is presented not merely as a novel composition, but as a structured, functional, and regulatory-aligned invention. The integration of dual-phase architecture, biomarker-adaptive logic, and validated delivery outcomes collectively support patentability across all statutory dimensions, anticipating and satisfying examiner expectations without reliance on reactive argumentation.

### Annex F—Daily Compound Composition by Functional Category

TABLE 1
Daytime Composition by Compound Category

| Compound Category | % Minimum DTD | % Maximum DTD |
|---|---|---|
| Functional Lipids | 20% | 50% |
| Bioactive Antioxidants | 3% | 15% |
| Regenerative Support | 1% | 8% |
| Molecules | | |
| Immune Modulators | 1% | 6% |
| Essential Minerals | 3% | 12% |
| Essential Fatty Acids | 3% | 12% |
| Bioactive Botanical | 3% | 12% |
| Compounds | | |

TABLE 2
Nighttime Composition by Compound Category

| Compound Category | % Minimum DTD | % Maximum DTD |
|---|---|---|
| Cell Signaling Complex | 1% | 12% |
| Immunomodulatory | 3% | 12% |
| Peptides | | |
| Regenerative Support | 1% | 8% |
| Molecules | | |
| Immune Modulators | 1% | 6% |
| Bioactive Antioxidants | 3% | 15% |
| Essential Fatty Acids | 3% | 12% |
| Essential Minerals | 3% | 12% |
| Bioactive Botanical | 3% | 12% |
| Compounds | | |
| Plant Adaptogens | 1% | 8% |

### Annex G—Structural and Functional Integration Statement

The present formulation integrates structural, functional, and timing-specific innovations not derivable from a mechanical combination of previously disclosed delivery systems. The biphasic architecture—comprising a sublingual liposomal morning-phase and a delayed-release hydrogel evening-phase—is governed by an embedded, non-therapeutic algorithm that synchronizes compound availability with circadian-phase metabolic receptivity.

These structural elements, when taken together, produce emergent delivery behavior characterized by:
Dual-peak nutrient alignment with biomarker trends (HRV, IL-6, cortisol:melatonin),
Bypassing of gastric degradation and hepatic first-pass metabolism during early-phase administration,
Coordinated systemic availability without invoking pharmacodynamic mechanisms.
   No known reference or combination of references discloses this interdependent structure, and no predictable modification of conventional systems yields the claimed synergy of release behavior, compound preservation, and regulatory alignment. Accordingly, the present system constitutes a structurally non-obvious, biomarker-modulated nutraceutical platform, operating fully within dietary supplement classification and eligible for protection under 35 U.S.C. §§ 101-103.

1. A biphasic circadian-aligned nutraceutical composition comprising:
(a) a first-phase formulation administered in the early diurnal period, comprising phosphatidylcholine-class phospholipids, adaptogenic phenolic glycosides, and stilbenoid-class polyphenols, each encapsulated within phospholipid-stabilized micellar vesicles, wherein the vesicles exhibit a particle size between 80-150 nm and a zeta potential ranging from −18 to −22 mV; and
(b) a second-phase formulation administered in the pre-nocturnal period, comprising phosphatidylserine-class lipids, non-proteinogenic amino acid analogs, and carotenoid-class antioxidants, each embedded in a pH-sensitive polysaccharide nanoparticle matrix with disintegration initiated below pH 5.8;
(c) wherein both phases are manufactured using high-pressure homogenization and lyophilization techniques, and exhibit bioavailability peaks at approximately 7±1 hours and 10±2 hours, respectively; and
(d) wherein the composition is composed entirely of GRAS-certified ingredients under 21 CFR § 170.30 and is aligned with general circadian physiological rhythms.

2. The composition of claim 1, wherein the micelles are stabilized using a phosphatidylcholine-cholesterol bilayer at a 4:1 molar ratio.

3. The composition of claim 1, wherein the polysaccharide nanoparticle matrix comprises a chitosan-alginate hydrogel co-lyophilized with tocopherol-class antioxidant stabilizers.

4. The composition of claim 1, wherein the micelles are generated using high-shear homogenization at 120 MPa.

5. The composition of claim 1, wherein enterocyte uptake is increased by at least 3.8-fold over unencapsulated equivalents, as validated through Caco-2 permeability assays.

6. The composition of claim 1, wherein enzymatic degradation is reduced by ≥42% based on intestinal and hepatic microsomal profiling via LC-MS/MS.

7. The composition of claim 1, wherein mitochondrial ATP production is enhanced through adaptogenic synergy, confirmed via oxygen consumption rate (OCR) analysis.

8. The composition of claim 1, wherein the disintegration of the second-phase matrix initiates at pH<5.5 within the proximal small intestine.

9. The composition of claim 1, wherein administration formats are selected from enteric-coated capsules, oral powder sachets, or sublingual hydrogel delivery systems.

10. The composition of claim 1, wherein the first-phase is administered within 0-2 hours post-awakening and the second-phase within 1-2 hours prior to sleep onset.

11. The composition of claim 1, wherein the composition supports physiological recovery and adaptive resilience in physically active users following intense exertion.

12. The composition of claim 1, wherein the second-phase is optimized for cellular energy management and mitochondrial function in users aged 60 years and older.

13. The composition of claim 1, adapted to promote immune equilibrium in individuals exposed to sustained occupational stress environments.

14. The composition of claim 1, wherein the composition is formulated for users subject to circadian rhythm disruption due to transmeridian travel or shift-based schedules.

15. The composition of claim 1, wherein the composition is included in a modular supplementation system with temporally separated AM and PM unit dosages configured for multi-day administration.

16. The composition of claim 1, wherein the first-phase is administered as a sublingual liposomal emulsion and the second-phase as a rehydratable hydrogel matrix, wherein such formats are structured to support non-invasive nutrient absorption, bypass gastric degradation and hepatic first-pass metabolism, and enable circadian-phase-aligned availability under nutraceutical use conditions.

17. A method for chrono-adaptive nutrient delivery, comprising:
(a) administering a morning-phase formulation comprising phospholipid-encapsulated adaptogens and antioxidant-class bioactives within two hours of waking;
(b) administering an evening-phase formulation comprising regenerative lipid mediators and neuro-supportive peptide complexes embedded in a pH-responsive nanoparticle matrix within two hours prior to sleep; and
(c) wherein the release timing is regulated by a non-therapeutic internal synchronization framework using biometric indicators such as heart rate variability (HRV), salivary cortisol, and IL-6 trends.

18. The method of claim 17, wherein the synchronization framework is implemented via a non-invasive biometric tracking device.

19. The method of claim 17, wherein nutrient release schedules are recalibrated weekly using biometric data including cortisol:melatonin ratio, HRV trends, and sleep cycle variability.

20. The method of claim 17, wherein delivery is executed using multilayer enteric capsules engineered for staged disintegration.

21. The method of claim 17, wherein the biometric-informed synchronization framework does not rely on therapeutic, diagnostic, or pharmacological feedback loops, and functions solely under GRAS-compliant modulation logic.

22. The method of claim 17, wherein the release behavior of both phases aligns with circadian receptivity windows without invoking systemic drug-like absorption kinetics.

23. A biphasic nutraceutical platform comprising:
(a) a first-phase formulation including stilbenoid polyphenols, mitochondrial coenzyme quinones, immunomodulatory polysaccharide fractions, and phenolic adaptogen derivatives, encapsulated in nanoemulsion micelles with mean size between 80-150 nm and zeta potential between −18 and −22 mV;
(b) a second-phase formulation including phosphatidylserine-class lipids, carotenoid antioxidants, polyphenolic diketones, non-proteinogenic amino acid analogs, long-chain polyunsaturated fatty acids, and adaptogenic peptides, embedded in a polysaccharide matrix with delayed disintegration below pH 5.8; and
(c) wherein the formulations are configured for synchronized bioavailability under GRAS-compliant dietary supplement production protocols.
